(19) United States
(12) Patent Application Publication
(10) Pub. No.: US 2022/0401030 A1
(43) Pub. Date: Dec. 22, 2022

(54) SHEATH OR CATHETER WITH DILATOR FOR TRANSSEPTAL PUNCTURE VISUALIZATION AND PERFORATION, AND METHOD OF USE THEREOF

(71) Applicant: NORTH STAR MEDICAL INC., Montréal, Québec (CA)

(72) Inventor: Olivier BATAILLE, Lachine (CA)

(21) Appl. No.: 17/763,710

(22) PCT Filed: Sep. 29, 2020
(86) PCT No.: PCT/CA2020/051296

§ 371 Date: Mar. 25, 2022

Related U.S. Application Data
(60) Provisional application No. 62/907,790, filed on Sep. 30, 2019.

(51) Int. Cl. A61B 5/00; A61B 18/24

(52) U.S. Cl. CPC A61B 5/6869; A61B 18/24; A61B 5/0036; A61B 5/0066; A61B 2018/00642

(57) ABSTRACT

A dilator used in performing a transseptal puncture; it has a hub with an opening at a proximal end; a shaft, connected to a distal end of the hub, comprising a lumen miming along a length of the shaft defining an inner wall within the shaft; an optical fiber for insertion into the lumen of the shaft, the optical fiber comprising a proximal end portion for sealing the opening of the hub, the optical fiber having a length for running along a length of the lumen; wherein the optical fiber is configured to, in a simultaneous or alternating fashion: propagate a laser beam with an ultrafast pulse duration that is generated by an ultrafast laser; and propagate light for obtaining visualization information from the light interacting with neighboring surfaces in the heart using optical coherence tomography.

The present application claims priority from U.S. provisional patent application No. 62/907,790 filed on Sep. 30, 2019, incorporated herein by reference.

### TECHNICAL FIELD

The present disclosure relates to sheaths and/or catheters, and more particularly to sheaths and/or catheters and/or dilator for use in minimally invasive cardiac procedures.

### BACKGROUND

The left atrium is the most difficult chamber of the heart to access via a minimally invasive approach. In the 1950's the transseptal procedure was developed to have a more direct access to the left atrium where many common interventional cardiology and electrophysiology procedures are performed. The Brockenbrough and Mullins devices and procedures evolved over time.

During beating heart minimally invasive cardiac surgery, the patient's cardiovascular system is accessed by the femoral vein and a transseptal puncture is practiced with a Brockenbrough curved needle brought to the puncture site in a sheath. It is not unusual to see that some patients returning for another procedure already had a transseptal puncture and scar tissue has formed on the previous puncture site, making it more difficult to use a standard Brockenbrough needle for a second transseptal puncture. Users can revert to a Baylis energy needle to overcome the difficulty of traversing the septum, however the Baylis technology is based on RF energy ablation and once the RF wire is passed through the septum it can easily travel to the other side of the left atrium and puncture the epicardial wall, creating a major bleed that may require surgery. It is also in general not easy to choose a transseptal site and perform the puncture in the intended site as the procedure is done on a beating heart.

Accordingly, it would be advantageous to provide systems, devices and methods by which the transseptal puncture can be safely performed by choosing the proper puncture site, securing the septum to perform the puncture at the chosen desired site and prevent damage to other tissues.

### SUMMARY

The present disclosure relates to equipment, systems and methods for performing minimally invasive surgical interventions on heart and other cardiovascular tissue where the surgery site is identified, visualized and secured, and where the extent of the procedure is monitored in order to minimise damage to other tissues.

A first broad aspect is a method for targeting the surgical intervention of the heart to a particular site through the use of imaging technology, e.g. optical coherence tomography, to visualize the tissue in real time, allowing the user to choose the optimal placement of the equipment for the procedure. A deflectable sheath or catheter is inserted through any entry point known in the art and extended through the circulatory system to the heart. An optical fiber runs along the length of the sheath or catheter and propagates amplified light for the purpose of imaging, e.g. by optical coherence tomography, where an OCT unit as is known in the art processes the data relating to the behaviour of the photons to produce a three-dimensional image of the target tissue. As such, the user can avoid targeting unsuitable tissue, e.g. scar tissue for intervention by being able to distinguish different density tissue such as healthy tissue or scar tissue. In some embodiments, the optic fiber may run through a lumen within a sheath and/or catheter and or dilator. In some embodiments, the optical fibre may be inserted into the sheath or catheter after the latter has been extended to the target tissue.

Another broad aspect is a method for securing the surgical equipment at the site of the intervention to prevent its displacement, e.g. by the movement of a beating heart.

Another broad aspect is a method for preventing damage to non-target tissues through the use of a continuous feedback loop to automatically shut off the surgical equipment once the procedure is complete.

Another broad aspect is a dilator used in performing a transseptal puncture. The dilator includes a hub with an opening at a proximal end; a shaft, connected to a distal end of the hub, comprising a lumen running along a length of the shaft defining an inner wall within the shaft; an optical fiber for insertion into the lumen of the shaft, the optical fiber comprising a proximal end portion for sealing the opening of the hub, the optical fiber having a length for running along a length of the lumen; wherein the optical fiber is configured to, in a simultaneous or alternating fashion: propagate a laser beam with an ultrafast pulse duration that is generated by an ultrafast laser; and propagate light for obtaining visualization information from the light interacting with neighboring surfaces in the heart using optical coherence tomography.

In some embodiments, the inner wall of the shaft and the optical fiber, once inserted into the shaft, may define an interior space in the lumen between the optical fiber and the inner wall of the shaft.

In some embodiments, the hub may include a vacuum port for connecting the dilator to a vacuum source and creating a vacuum in the inner unoccupied space.

In some embodiments, the proximal end portion for sealing the opening of the hub may have a luer configuration that interacts with a sealing portion at the proximal end of the hub.

In some embodiments, the distal end of the shaft may have a tapered tip.

In some embodiments, the optical fiber may be a dual-core optical fiber, and wherein the laser beam may be propagated in a first core of the dual-core fiber, and wherein the light may be propagated in a second core of the dual-core fiber.

In some embodiments, the laser beam may be a Gaussian beam.

Another broad aspect is a kit used in the performance of a transseptal puncture. The kit includes the dilator as defined herein; a sheath including a shaft; a pull-wire assembly comprising one or more pull wires connected to a distal end of the shaft of the sheath; a steering mechanism connected to the one or more pull wires for causing tension to be applied to or diminished from one or more of the one or more pull wires for steering the shaft or catheter; and an opening providing access to a space for receiving the dilator.

In some embodiments, the dilator may be received in the space, and wherein one or more snap features may be used to secure the dilator to the sheath.

In some embodiments, the sheath further may include a handle at or near the proximal end of the sheath, wherein a wheel of the steering mechanism, for causing tension to be applied to or diminished from one or more of the one or more pull wires, may be located at the handle.

In some embodiments, the sheath may include, at or near the proximal end, a hemostatic valve body comprising the opening at a proximal end of the hemostatic valve body.

In some embodiments, the hemostatic valve body may include a hemostatic valve.

Another broad aspect is a system for performing a transseptal puncture. The system includes the kit as defined herein; one or more light sources for generating the laser beam and the light; a power source for powering the light source; and a controller configured to receive, at least periodically during the transseptal puncture, the light information and perform optical coherence tomography using the light information to obtain the visualization information; and at least periodically adapt, during the transseptal puncture, one or more properties of the laser beam as a function of the visualization information, the properties of the laser beam including pulse duration, wavelength, light source of the laser beam, and turning on or off a light source of the one or more light sources that generates the laser beam.

In some embodiments, the hub of the dilator further may include a vacuum port for connecting the dilator to a vacuum source and creating a vacuum in the inner unoccupied space, and wherein the system may include the vacuum source.

In some embodiments, the system may include a display for viewing the visualization information.

In some embodiments, the controller may be further configured to detect, using the visualization information, when the septum has been traversed, and to shut off a light source of the one or more light sources that generates the laser beam.

Another broad aspect is a method of puncturing heart tissue of a heart during a cardiac procedure including exposing heart tissue to a laser beam with an ultrafast pulse duration generated by an ultrafast laser in order to puncture the heart tissue.

In some embodiments, the heart tissue that is exposed to the laser beam may be that of a septum.

In some embodiments, the method may include directing light to surfaces of the heart to obtain light information for use in performing optical coherence tomography in order to obtain visualization information during the exposing.

In some embodiments, the laser beam and the beam of light may be propagated using the same optical fiber.

In some embodiments, one or more properties of the laser beam may be adapted, during the exposing, as a function of the visualization information, wherein the properties include pulse duration, wavelength, light source of the laser beam, and turning on or off a light source of the one or more light sources that generates the laser beam.

In some embodiments, the method may include shutting off a light source generating the laser beam when the heart tissue is punctured, the puncturing monitored through the visualization information.

In some embodiments, the method may include detecting scar tissue using the optical coherence tomography.

In some embodiments, the method may include applying a vacuum to remove debris during the cardiac procedure.

In some embodiments, the method may include applying a vacuum to secure the heart tissue to a tip of a dilator that has received an optical fiber that is adapted to propagate the laser beam.

In some embodiments, the method may include applying a vacuum to improve the visualization information generated using optical coherence tomography by removing blood near tissue to which the light is directed.

In some embodiments, the method may include, prior to the exposing, inserting a dilator, configured to receiving an optical fiber, into a sheath for guiding a distal tip of the dilator to a puncture site comprising the heart tissue.

In some embodiments, the method may include securing the dilator to the sheath.

Another broad aspect is a method for preparing for performing a transseptal puncture comprising inserting a dilator into a sheath for guiding a distal tip of the dilator, and further inserting an optical fiber into a shaft of the dilator, such that the optical fiber runs along a length of the shaft of the dilator, to a puncture site comprising the heart tissue.

Another broad aspect is use of an optical fiber for propagating a laser beam with an ultrafast pulse duration to a puncture site in heart tissue to conduct a transseptal puncture through an a-thermal process to reduce or eliminate the presence of scar tissue resulting from conducting the puncture.

Another broad aspect is use of an optical fiber for propagating a laser beam with an ultrafast pulse duration to a puncture site in heart tissue to conduct a transseptal puncture through an a-thermal process to reduce or eliminate the presence of scar tissue resulting from conducting the puncture; and propagating light to surfaces of a heart to obtain light information that is used in optical coherence tomography for obtaining visualization information during the transseptal puncture.

Another broad aspect is a dilator used in performing a transseptal puncture. The dilator includes a hub with an opening at a proximal end; a shaft, connected to a distal end of the hub, comprising a lumen running along a length of the shaft defining an inner wall within the shaft; an optical fiber for insertion into the lumen of the shaft, the optical fiber comprising a proximal end portion for sealing the opening of the hub, the optical fiber having a length for running along a length of the lumen; wherein the optical fiber is a dual-core optical fiber that is configured to, in a simultaneous or alternating fashion: propagate a laser beam with an ultrafast pulse duration that is generated by an ultrafast laser in a first core of the dual-core optical fiber; and propagate light in a second core of the dual-core optical fiber for obtaining visualization information from the light interacting with neighboring surfaces in the heart using optical coherence tomography.

### BRIEF DESCRIPTION OF THE DRAWINGS

The invention will be better understood by way of the following detailed description of embodiments of the invention with reference to the appended drawings, in which:

FIG. 1 is a schematic illustration of a cross-section of a human heart;

FIG. 2 is an illustration of a cross-section of a human heart showing the first steps of a traditional technique for performing an exemplary transseptal puncture to access the septum;

FIG. 3 is an illustration of cross-sections of a human heart and exemplary steps for performing an exemplary transseptal puncture with a dilator traversing the septum;

FIG. 4 is an illustration of an exemplary system for performing an advanced transseptal puncture;

FIGS. 5A, 5B and 5C, collectively referred to as FIG. 5, are illustrations of exemplary single patient use sheath, dilator and optical fiber respectively to perform an advanced transseptal puncture;

FIG. 6 is an illustration of an axial cross section of the tip of an exemplary dilator coupled with an exemplary optical fiber for the perforation of the septum;

FIG. 7 is an illustration of an axial cross section of the tip of an exemplary dilator coupled with an exemplary optical fiber in position for advanced transseptal puncture;

FIG. 8 is a flowchart diagram of an exemplary method of conducting a transseptal perforation using an ultrafast laser; and

FIG. 9 is a block diagram of an exemplary system for conducting visualization and performing transseptal perforation and/or a surgical intervention on heart tissue.

### DETAILED DESCRIPTION

In the present disclosure, by “surgical intervention of the heart”, it is meant a procedure that involves the removal or reshaping of heart tissue.

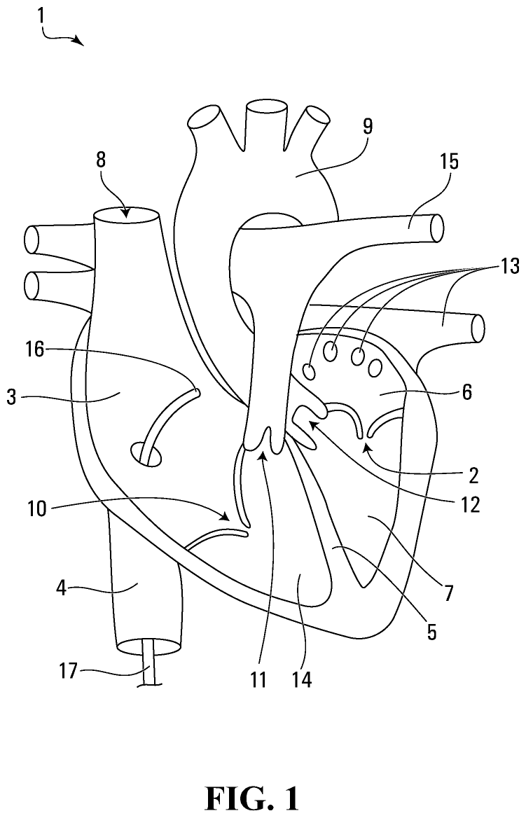

FIG. 1 shows a two-dimensional cross section of a typical human heart. FIG. 1 is referred to herein to better describe where the deflectable sheath 17, dilator 18, optical fiber 19 and guidewire 59 may be positioned. Embodiments described herein may be specifically designed to perform a transseptal puncture. The devices may be designed to be used in a minimally invasive surgery where the initial entry point can be the femoral vein in the groin, and where the deflectable sheath may be advanced through the inferior vena cava 4, into the right atrium 3. Once in the right atrium 3 the device can be used to perform a transseptal puncture.

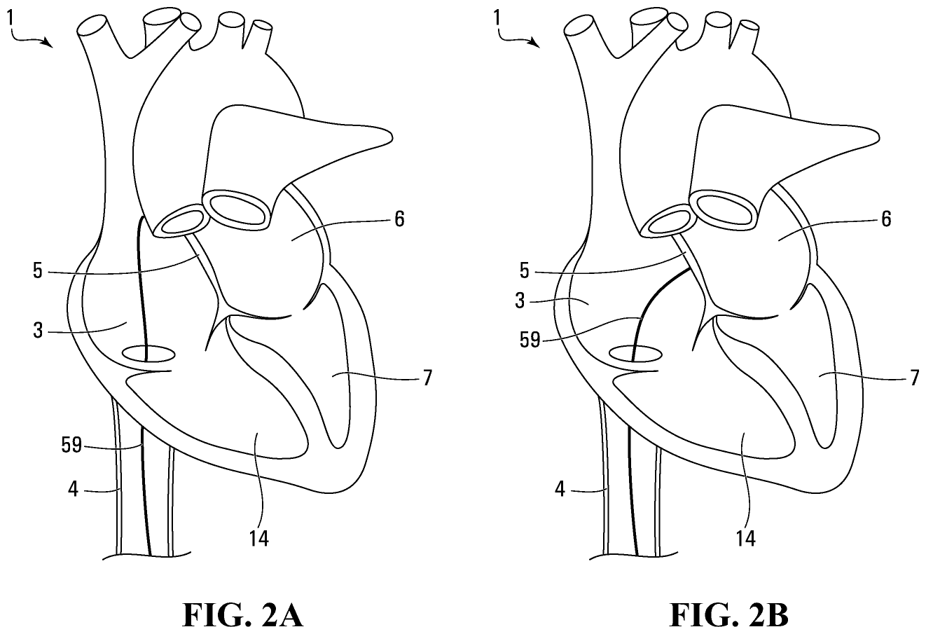

Reference is now made to FIG. 2 illustrating the initial steps of an exemplary access to the septum for the purpose of performing a transseptal puncture. A transseptal deflectable sheath is placed into the inferior vena cava 4 and the dilator with guidewire is advanced into the superior vena cava 8. The sheath is pulled down and the dilator tip and guidewire rest on the septum.

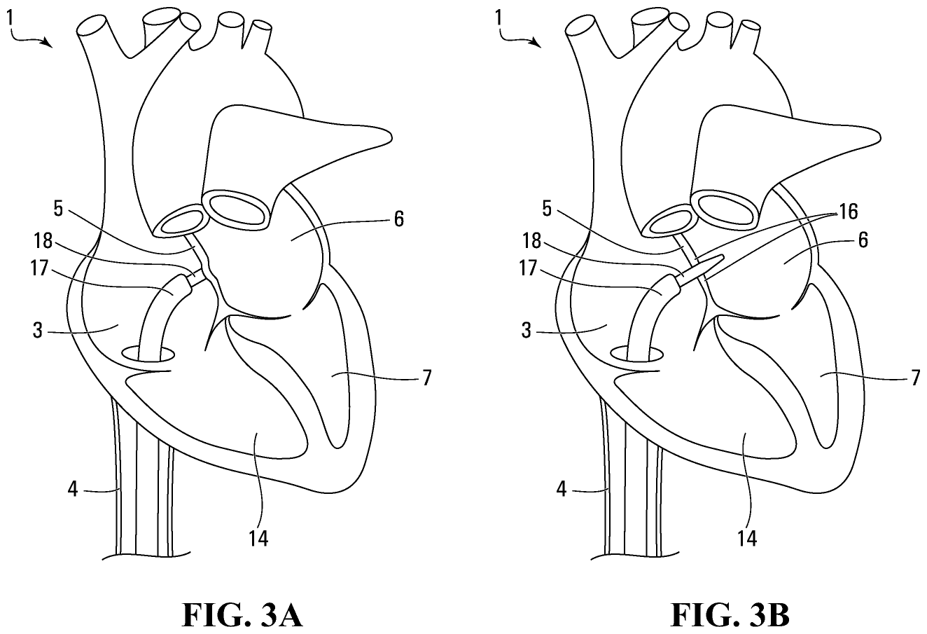

Reference is now made to FIG. 3 illustrating the puncturing of the septum with either a Brockenbrough needle, an RF wire or a laser puncture with imaging. The guidewire is replaced by the optical fiber that can perform the dual function of imaging with optical coherence tomography and puncturing the septum with a laser. In some instances, the laser is an ultrafast laser and performs the puncture a-thermally. In some embodiments, the laser beam is a Gaussian beam.

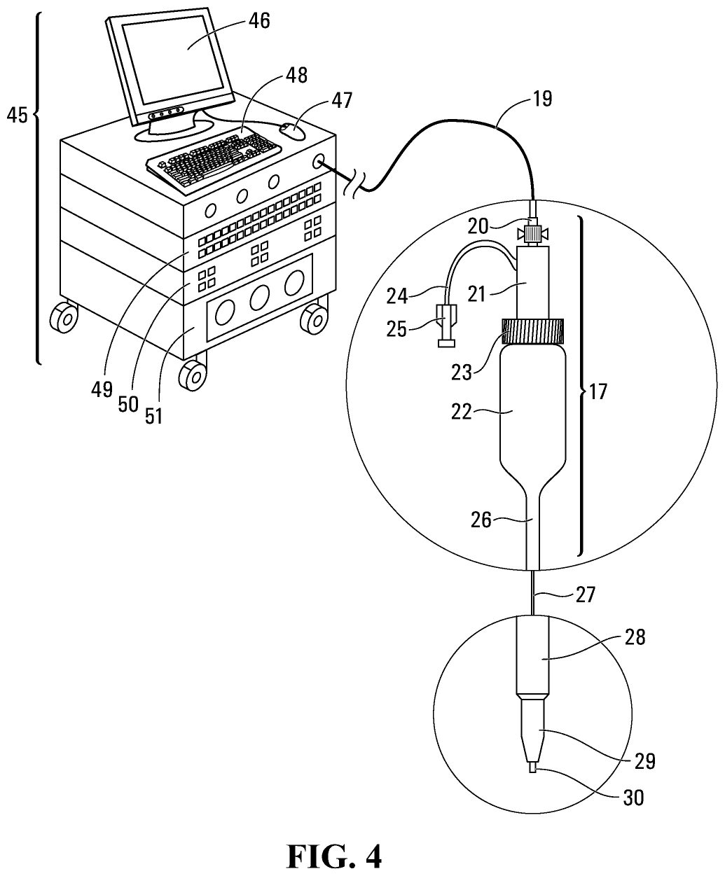

Reference is now made to FIG. 4 illustrating an exemplary imaging and perforating system for the crossing of the septum wall with virgin septum or scar septum. The system may have a deflectable sheath that may be advanced near the septum to be perforated and the dilator and optical fiber can be guided to the septum with a deflection mechanism sheath handle 22 where, by actuating the deflection mechanism 23, one or more pull wires are pulling on the pull wire ring assembly to deflect the sheath tip so it can reach the proper location on the septum. The optical fiber and dilator may be connected to the system console that comprises a vacuum pump and its controls 51, an ultrafast laser with harmonic generator and its controls 50, an OCT light source, its controls and a PC 49, a monitor 46, and a user input interface (e.g. a mouse 47 and a keyboard 48). Once tip 29 of the dilator is in contact with the septum, the optical fiber inner core is illuminated and with a standard OCT system to provide an in-depth image of the septum to confirm proper location. Once proper location has been confirmed, the vacuum pump 51 is turned on and a vacuum is generated so that the tip of the dilator adheres to the septal tissue by suction to secure and not lose the proper location. Once the system has secured the septum, OCT module 49 is powered on again so that its light source is conveyed via the inner core optical fiber 55 so the system 45 can display real time tomography on the monitor 46. As it is displaying real time tomography, it is mapping the three-dimensional construction of the septum at that location. The operator can decide to set the system parameters manually for tissue pulverization function or to use the integrated algorithm, based on the OCT, to set the ultrafast laser to the optimal setting for the particular site of the pulverisation puncture. The setting can also determine the optimal wavelength to safely pulverize the septum tissue. The system can also be used for any other pulverization or puncture medical procedures. Once the system has been properly set to the desired pulverisation mode, the ultrafast laser is actuated and the ultrafast laser beam is carried to the treatment site with the coaxial optical fiber outer core 54. As it is difficult for the OCT technology to penetrate tissue with blood in between, not only does the vacuum suction lumen 56 provide a means to secure the septum, it also permits the OCT system to fully penetrate the septum wall for the 3D imaging and scar tissue detection. Furthermore, the vacuum lumen permits the evacuation of the pulverized nano-particles safely.

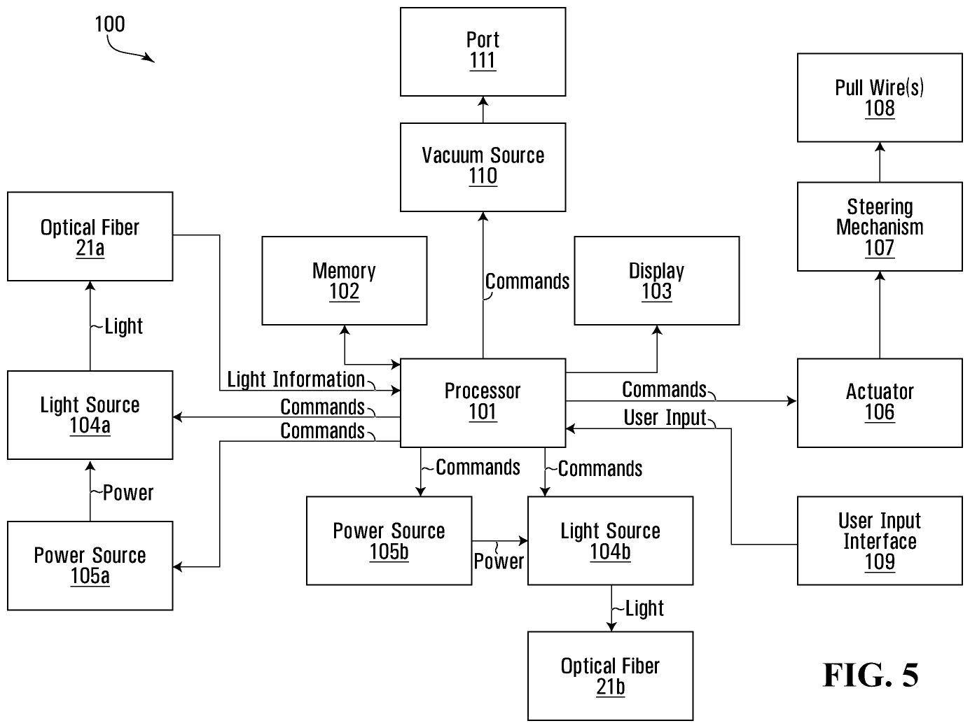

Referring now to FIGS. 5a, 5b, and 5c, an exemplary deflectable sheath 17 with an exemplary dilator 18 and an exemplary optical fiber 19 that are designed to fit into one another and provide a proper focal point for the OCT and the pulverization laser beam are shown. The exemplary deflectable sheath 17 has a standard deflection mechanism with a sheath deflection knob 23, a handle 22, a shaft 27, a clear hemostatic valve body 21, a hemostatic valve 38 that may be housed in the clear hemostatic valve body 21, a side luer tubing 24 with a luer port 25 (e.g. for receiving a saline and/or heparin drip to, e.g., create positive pressure to prevent air ingress and/or to thin the blood to prevent blood clot). The proximal side of the clear hemostatic valve body 21 also has a snap feature 31 so the dilator hub 20 can snap in place once inserted into the sheath by having a matching snap feature 32. The dual core optical fiber 19 can be inserted into the dilator and the optical fiber is secured in the dilator at the proper and constant distance by having matching luer type male/female seals 35 and 36 to which they can be secured. In other examples, other mechanisms can be used to secure the optical fiber to the dilator and to seal the optical fiber therein in order to avoid air ingress, such as by securing a cap for sealing the optical fiber to the dilator. The location of the optical fiber may be such that the distal tip of the optical fiber is recessed, e.g., a few microns, from the tip of the dilator, in order to optimize the focal distance to the target tissue. Furthermore, the feature provides an airtight lock between the dilator and the dual core optical fiber. Once the device is properly placed on the septum, the dilator hub vacuum side port 57, already connected to the console, can provide a vacuum all the way to the tip and secure the tissue by suction. In other embodiments, the vacuum may be provided through vacuum lumen 56.

In some embodiments, the optical fiber may be a dual body optical fiber including an inner core and an outer core, wherein the outer core of the dual body optical fiber may be a hollow core optical fiber, wherein the visualization may be carried out by the inner core, and the pulverizing of heart tissue may be performed by photon energy transported by the outer core.

In some embodiments, the dual function may be achieved with a multimode optical fiber for propagating the laser beam and the light.

In some embodiments, the dilator may have two lumens for receiving two separate optical fibers, where one optical fiber transports the laser beam, and the other fiber transports the light.

It will be understood that other configurations of the optical fiber may be used to both propagate the laser and the light used for generating the visual information.

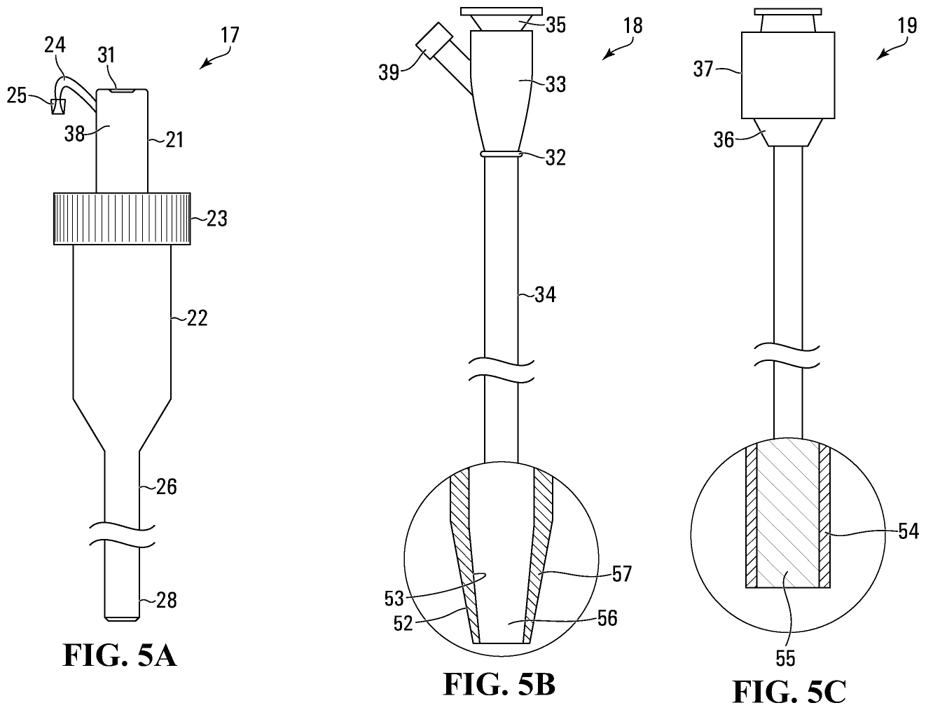

Referring now to FIG. 6, an exemplary dilator tip 29 comprising outer wall 52, inner wall 53, vacuum lumen 56 and side port 57, wherein is inserted an optical fibre comprising an outer core 54 and inner core 56.

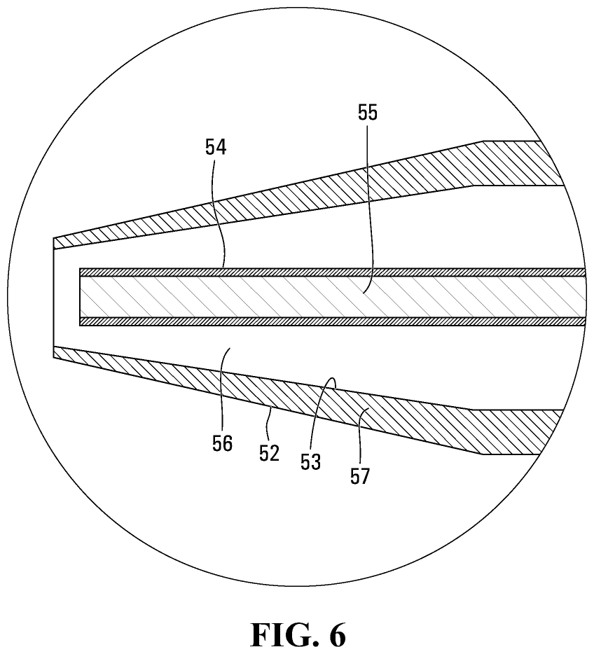

Referring now to FIG. 7, an exemplary dilator tip 29, containing an optical fiber consisting of outer core 54 and inner core 55, adhering to septum 5 by suction, is shown, the vacuum being provided through either vacuum lumen 56 or side port 57. An imaging laser or superluminescent diode may be directed through the optical fiber inner core 55 and optical coherence tomography imaging may be used to verify proper placement of the dilator tip and optical fiber onto the septum. In the event that the dilator tip is improperly placed, the placement can be adjusted. A laser beam of an ultrafast laser is then carried through the optical fiber for tissue pulverisation.

The ultrafast laser is a laser capable of transmitting ultrafast pulses, e.g., pico-and/or femtosecond pulses, where use of the ultrafast laser may result in an a-thermal, or a nearly a-thermal process (considered, in the present disclosure, as being a-thermal).

The ultrafast laser may have an optical fiber (which includes a cable composed of optical fibers) for delivering the light beam. Optical fibers used for beam delivery of an ultrafast laser are known in the art. For instance, reference is made to Bjorn Wedel and Max Funck, “Industrial Fiber Beam Delivery System for Ultrafast Lasers”, Laser Technik Journal, April 2016, pages 42 to 44, where an optical fiber with a hollow core structure is described. The micro-structure hollow core fibers support light propagation inside the hollow core (e.g. in a gas or vacuum). However, it will be understood that other optical fibers may be used to propagate a laser beam for an ultrafast laser without departing from the present teachings.

In some exemplary embodiments, the ultrafast laser may include a laser source, the optical fiber, and a coupling unit for adapting the size of the beam and focusing the laser beam to the tip of the optical fiber.

In some examples, the optical fiber used for visualization may also be used as an ultrafast laser for performing other surgical intervention carried out on heart tissue. In these examples, for instance, the optical fiber may be a dual body fiber, one with an outer core and inner where the inner core can convey the light used for visualization and the outer core may convey the photon energy. In other examples, the optical fiber may include a dual path fiber. In some examples, the device can alternate between photon emission and imaging. In some examples, the energy reflection of the ultrafast laser function can be used as a light source used for performing optical coherence tomography.

In some examples, the sheath, catheter and/or dilator used for a transseptal puncture may include a lumen to receive an optical fiber used for pressure measurement than can be inserted into the lumen. An exemplary optical fiber for pressure measurement is described in U.S. patent application Ser. No. 13/834,746, incorporated herein by reference.

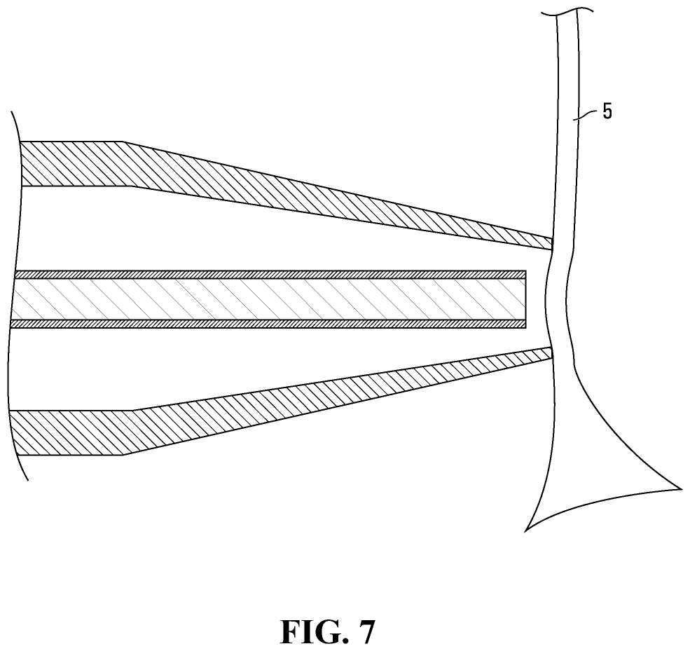

Reference is now made to FIG. 8, illustrating an exemplary method 1500 for monitored septum puncture by pulverisation of the tissue and/or performing a surgical intervention on heart tissue.

A transseptal deflectable sheath is placed into the inferior vena cava 4. A dilator with a guidewire is inserted in the sheath. The dilator with guidewire is advanced into the superior vena cava 8 at step 1510. In some embodiments, the sheath may be pulled down and the dilator tip and guidewire rest on the septum at step 1520.

The guidewire may then be removed from the dilator. An optical fiber is inserted into the patient at step 1530, in some embodiments, in the place of the guidewire, sliding into a lumen of the dilator such that the tip of optical fiber may rest only a few microns from the tip of the dilator such that the focal distance of the target tissue is only, e.g., a few microns. The optical fiber may be secured to the dilator such than an air-tight seal is achieved to avoid air ingress (e.g. using the luer snap feature described herein). The optical fiber may be used to propagate light from a light source to the site of the cardiac procedure, the light exiting the optical fiber and projecting onto heart tissue. Visualization information is obtained from the behavior of the light as it reaches surrounding surfaces (e.g. heart tissue) through optical coherence tomography at step 1540.

The visualization information is used to adjust the ultrafast laser properties, such as its position, its pulse duration, wavelength, focal distance, laser source, etc., based, e.g., on the properties of the site of the surgical intervention (e.g. size, density, tissue properties, distance separating exit point of laser beam and target site for pulverization and/or surgical intervention, etc.) at step 1550.

The laser is then generated to pulverize the septum tissue and/or perform the surgical intervention (e.g. a-thermal ablation, cutting, etc.), exiting the tip of the laser beam, directed to the target tissue, at step 1560.

During the laser process, visualization information may be regularly generated by the light information (the light being generated by the light source during the laser processing), providing feedback information on the laser processing at step 1570.

In some embodiments, a vacuum may also be created to remove blood surrounding the heart tissue to be visualized, the removal of blood facilitating the visualization.

In some examples, a vacuum may also remove pulverized particles and debris, to, e.g., avoid an embolism.

In some embodiments, a vacuum may be used to secure the heart tissue to the tip of the dilator.

The feedback visualization information may be used to determine if the septum has been punctured at step 1580, or if properties of the laser beam may be adjusted during the procedure (e.g. as a function of the progress of the procedure to determine if, e.g., the septum is almost punctured).

If the procedure is not complete at step 1595, as a function of the visualization information, additional properties of the ultrafast laser may be adjusted at step 1540, where steps 1540-1570 are repeated until the procedure is complete.

If the septum has been punctured at step 1590, the laser may be turned off at step 1600.

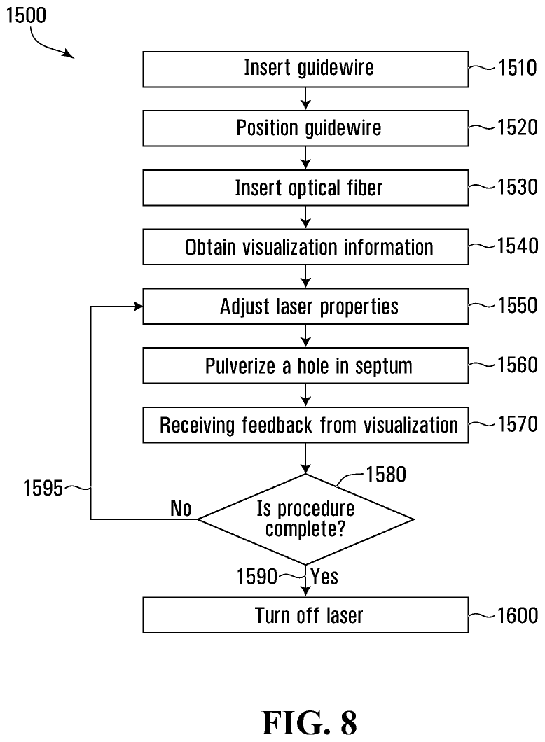

Reference is now made to FIG. 9, illustrating an exemplary system 100 for pulverizing heart tissue (e.g. for performing a transseptal puncture) and/or for performing a surgical intervention targeting heart tissue.

The system 100 includes a processor 101, memory 102, a power source 105b for powering a laser source 104b, an optical fiber 21b for propagating a laser beam generated by the laser source 104b, a power source 105b for powering a light source 104a, and an optical fiber 21a for propagating light from the light source 104a.

The system 100 may have an actuator 106 for, e.g., electrically, mechanically or pneumatically controlling the steering mechanism 107 of a deflectable sheath or catheter, the steering mechanism 107 causing deflection of the tip of a shaft of the sheath or catheter by applying or removing tension from the one or more pull wires 108 of the sheath or catheter.

The system 100 may have a user input interface 109 and a display 103.

The processor 101 and memory 102 may be connected via, e.g., a BUS, where the processor 101 carries out instructions by executing program code stored in the memory 102.

The memory 102 is a storage medium for storing program code and data that is retrievable by the processor 101.

The processor 101 and the memory 102 may be referred to herein as a controller.

The user input interface 109 receives input from a user to, e.g., turn on/off power source 105a, power source 106b, adjust the properties of the laser source 104b, control the steering mechanism 107 via the actuator 106, etc. The user input interface 109 may be, e.g., a touchscreen, a keyboard, a mouse, a microphone, a button, etc.

The display 103 may be a screen for showing certain images to the user, such as the image of the surgical site generated by optical coherence tomography, allowing the user to, e.g., view the progress of the decalcification or surgical intervention.

The steering mechanism 107 may be integrated or present in the handle of the catheter/sheath. The steering mechanism may be integrated or part of a robot that is computer-controlled, such as a surgical robot as is known in the art.

The one or more pull wires 108 are located in the shaft of the catheter and/or sheath, and attached to or near the distal end of the shaft. The properties of the one or more pull wires 108 and the positioning of the one or more pull wires 108 within the shaft of the catheter or sheath are as is known in the art for a deflectable catheter or sheath.

The power source 105a (e.g. an electrical outlet, a battery, etc.) provides power to the light source 104a. The light source 104a generates light that is propagated by the optical fiber 21a.

In the present disclosure, by optical fiber, it is meant an optical fiber or a bundle of optical fibers that may be encased in a housing (e.g. forming a cable).

The optical fiber 21a projects light on a nearby surface to conduct a surgical intervention or pulverization. Light reflection is then used to provide information to the processor 101 to conduct visualization of the site using optical coherence tomography. Optical coherence tomography may be achieved by using processes as are known in the art.

Power source 105b (e.g. an electrical outlet, a battery, etc.) provides power to the laser source 104b. The laser source 104b may be one as is known in the art to provide an ultrafast laser beam (at or below a few picoseconds pulse durations, where the processing by the laser beam is an a-thermal process). The laser beam produced by the laser source 104b may then be propagated by the optical fiber 21b to the target site for pulverizing and/or conducting the surgical intervention on heart tissue.

It will be understood that there may be a single power source 105 for powering light source 104a and laser source 105b. There may be a single light or laser source 104 and optical fiber 21 for generating and propagating photons for either visualization or laser processing (e.g. cutting, pulverization), where, e.g., the properties of the laser source 104 may be adapted by the processor 101 as a function of the desired function (visualization or laser processing). The optical fiber 21 may consist of separate cores for propagating photons from different sources.

During the course of the pulverization and/or the surgical intervention, the processor 101 may generate data using optical coherence tomography, based on the light information provided by the optical fiber 21a, to further adjust the properties of the laser source 104b, such as the pulse duration, the light wavelength, etc., or to change the laser source 104b.

In some embodiments, the data generated by the processor 101 using optical coherence tomography may be used to obtain depth information pertaining to the site of the transseptal puncture or the site of the surgical intervention. The processor 101 may then generate commands directed to the laser source 104b to modify, e.g., the laser focal distance or to shut off the laser.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

### Numerals Referred to in the Figures

1) Heart
2) Mitral Valve
3) Right atrium
4) Inferior vena cava
5) Septum
6) Left atrium
7) Left ventricle
8) Superior vena cava
9) Aorta
10) Tricuspid valve
11) Pulmonary valve
12) Aortic Valve
13) Pulmonary veins
14) Right ventricle
15) Pulmonary artery
16) Valve leaflet
17) Deflectable sheath
18) Dilator
19) Coaxial optical fiber
20) Dilator hub
21) Deflectable Sheath clear hemostatic valve body
22) Deflectable sheath handle
23) Sheath deflection knob or thumb wheel
24) Sheath side port tubing
25) Sheath side port luer hub
26) Deflectable sheath shaft proximal
27) Deflectable sheath shaft
28) Deflectable sheath shaft distal
29) Dilator tip
30) Coaxial optical fiber tip
31) Hemostatic valve snap feature
32) Dilator hub snap feature
33) Dilator hub
34) Dilator shaft
35) Dilator luer seal feature
36) Optical fiber connector luer seal feature
37) Optical fiber luer seal
38) Sheath Hemostatic valve
39) Dilator hub vacuum side port
45) Console (Controls, keyboard, mouse, monitor, Vacuum, Ultrafast fiber laser, OCT light source, harmonic generator,
46) Monitor
47) Mouse
48) Keyboard
49) OCT light source, controls, PC,
50) Ultra fast laser and controls
51) Vacuum pump an controls
52) Dilator shaft outer wall
53) Dilator shaft inner wall
54) Coaxial Optical fiber outer core
55) Coaxial Optical fiber inner core
56) Dilator shaft vacuum lumen
57) Dilator side port
58) Dilator deflection knob or thumb wheel
59) Standard Guidewire

What is claimed is:

1. A dilator used in performing a transseptal puncture comprising:
a hub with an opening at a proximal end;
a shaft, connected to a distal end of the hub, comprising a lumen running along a length of the shaft defining an inner wall within the shaft;
an optical fiber for insertion into the lumen of the shaft, the optical fiber comprising a proximal end portion for sealing the opening of the hub, the optical fiber having a length for running along a length of the lumen;
wherein the optical fiber is configured to, in a simultaneous or alternating fashion:
propagate a laser beam with an ultrafast pulse duration that is generated by an ultrafast laser; and
propagate light for obtaining visualization information from the light interacting with neighboring surfaces in the heart using optical coherence tomography.

2. A kit used in the performance of a transseptal puncture comprising:
the dilator as defined in claim 1;
a sheath comprising:
a shaft;
a pull-wire assembly comprising one or more pull wires connected to a distal end of the shaft of the sheath;
a steering mechanism connected to the one or more pull wires for causing tension to be applied to or diminished from one or more of the one or more pull wires for steering the shaft or catheter; and
an opening providing access to a space for receiving the dilator.

3. A system for performing a transseptal puncture comprising:
the kit as defined in claim 2;
one or more light sources for generating the laser beam and the light;
a power source for powering the light source; and
a controller configured to:
receive, at least periodically during the transseptal puncture, the light information and perform optical coherence tomography using the light information to obtain the visualization information; and
at least periodically adapt, during the transseptal puncture, one or more properties of the laser beam as a function of the visualization information, the properties of the laser beam including pulse duration, wavelength, light source of the laser beam, and turning on or off a light source of the one or more light sources that generates the laser beam.

4. The system as defined in claim 3, wherein the hub of the dilator further comprises a vacuum port for connecting the dilator to a vacuum source and creating a vacuum in the inner unoccupied space, and wherein the system further comprises the vacuum source.

5. The system as defined in claim 3 or claim 4, wherein the controller is further configured to detect, using the visualization information, when the septum has been traversed, and to shut off a light source of the one or more light sources that generates the laser beam.

6. A method of puncturing heart tissue of a heart during a cardiac procedure comprising:
exposing heart tissue to a laser beam with an ultrafast pulse duration generated by an ultrafast laser in order to puncture the heart tissue.

7. The method as defined in claim 6, further comprising directing light to surfaces of the heart to obtain light information for use in performing optical coherence tomography in order to obtain visualization information during the exposing.

8. The method as defined in claim 7, wherein the laser beam and the beam of light are propagated using the same optical fiber.

9. The method as defined in claim 7 or claim 8, wherein one or more properties of the laser beam are adapted, during the exposing, as a function of the visualization information, wherein the properties include pulse duration, wavelength, light source of the laser beam, and turning on or off a light source of the one or more light sources that generates the laser beam.

10. The method as defined in claim 9, further comprising shutting off a light source generating the laser beam when the heart tissue is punctured, the puncturing monitored through the visualization information.

11. The method as defined in any one of claims 7 to 10, further comprising detecting scar tissue using the optical coherence tomography.

12. The method as defined in any one of claims 6 to 11, further comprising applying a vacuum to at least one of:
remove debris during the cardiac procedure;
secure the heart tissue to a tip of a dilator that has received an optical fiber that is adapted to propagate the laser beam; and
improve the visualization information generated using optical coherence tomography by removing blood near tissue to which the light is directed.

13. A method for preparing for performing a transseptal puncture comprising inserting a dilator into a sheath for guiding a distal tip of the dilator, and further inserting an optical fiber into a shaft of the dilator, such that the optical fiber runs along a length of the shaft of the dilator, to a puncture site comprising the heart tissue.

14. Use of an optical fiber for:
propagating a laser beam with an ultrafast pulse duration to a puncture site in heart tissue to conduct a transseptal puncture through an a-thermal process to reduce or eliminate the presence of scar tissue resulting from conducting the puncture; and
propagating light to surfaces of a heart to obtain light information that is used in optical coherence tomography for obtaining visualization information during the transseptal puncture.
